(19) United States
(12) Patent Application Publication
(10) Pub. No.: US 2006/0173174 A1
(43) Pub. Date: Aug. 3, 2006

(54) Difluoronucleosides and process for preparation thereof

(76) Inventors: Anne-Ruth Born, Basel (CH); Pierre Martin, Rheinfelden (CH); Dirk Spielvogel, Freiburg (DE); Marco Villa, Milano (IT)

Correspondence Address: KENYON & KENYON LLP, ONE BROADWAY, NEW YORK, NY, 10004, US

(21) Appl. No.: 11/298,359

(22) Filed: Dec. 8, 2005

Related U.S. Application Data
(60) Provisional application No. 60/634,376, filed on Dec. 8, 2004.

(51) Int. Cl. C07H 19/048; C07F 9/6512

(52) U.S. Cl. 536/28.1; 544/243; 536/55.3

(57) ABSTRACT

A stereoselective process for the preparation of a 2′,2′-difluronucleoside is provided. In the process, a protected 2′2′-difluorofuranose is coupled with a base selected from the group consisting of pyrimidine and purine derivatives in the presence of a Lewis acid, wherein the protected 2′2′-difluorofuranose has a 1-position leaving group and 3- and 5-position protecting groups, and, when the base comprises 1 or more oxygen atoms, the base is a protected base, wherein each oxygen atom is protected with a protecting group.

### RELATED APPLICATIONS

This application claims the benefit of U.S. provisional application No. 60/634,376, filed Dec. 8, 2004, hereby incorporated by reference.

### FIELD OF INVENTION

The invention is directed to the novel difluoronucleoside, 2-deoxy-3,5-dibenzoate-2,2-difluoro-uridine, and to the process for preparation thereof.

### BACKGROUND

Gemcitabine HCl is the beta isomer of 2′-deoxy-2′,2′-difluorocytidine monohydrochloride, having the following structure

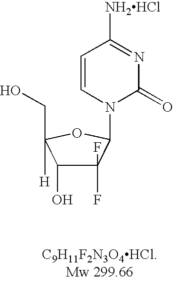

It is a white to off-white solid, marketed under the name Gemzar® as a nucleoside analogue that exhibits antitumor activity. Gemcitabine, which is the free base of Gemcitabine hydrochloride, exhibits cell phase specificity, primarily killing cells undergoing DNA synthesis (S-phase), and also blocking the progression of cells through the G1/S-phase boundary. Gemcitabine is metabolized intracellularly by nucleoside kinases to the active diphosphate (dFdCDP) and triphosphate (dFdCTP) nucleosides. The cytotoxic effect of gemcitabine is attributed to a combination of two actions of the diphosphate and the triphosphate nucleosides, which leads to inhibition of DNA synthesis.

Gemcitabine hydrochloride is prepared from Gemcitabine, which is a 2′,2′-difluoronucleoside derivative that is usually prepared by the attack of a suitable protected base on the 1-position of a corresponding protected sugar derivative.

U.S. Pat. No. 4,965,374 discloses the coupling reaction between 1-sulphonyloxy-2-deoxy-2,2,-difluoropentofuranoses and a protected cytidine, to yield the precursor of Gemcitabine as a mixture of cc/p isomers in a ratio of 1:1.

U.S. Pat. No. 5,371,210 discloses the coupling reaction between 1-sulphonyloxy-2-deoxy-2,2,-difluoropentofuranoses and a protected cytidine, but the reaction is carried out without any solvent. However, a pre-purification process of the 1-sulphonyloxy-2-deoxy-2,2,-difluoropentofuranoses is conducted to obtain an isomerically enriched starting material, that after the coupling reaction leads to the precursor of Gemcitabine having an α/β ratio of up to 1 to 1.8.

U.S. Pat. No. 5,594,124 discloses the coupling reaction between 1-sulphonyloxy-2-deoxy-2,2,-difluoropentofuranoses and a protected cytidine at −78° C., giving the final product with an α/β ratio of up to 1 to 2.5.

U.S. Pat. No. 5,744,597 discloses the coupling reaction between 1-sulphonyloxy-2-deoxy-2,2,-difluoropentofuranoses and a protected cytidine, after a pre-purification process, as described in U.S. Pat. No. 5,371,210.

The US Pharmacopoeia sets a very strict limitation on the level of the a isomer in Gemcitabine (the β isomer), thus allowing a level of no more than 0.1% area by HPLC. Therefore, there is a need in the art for an improved process for the preparation of 2′,2′-difluoronucleosides.

### SUMMARY OF THE INVENTION

In one aspect, the present invention provides a process for the preparation of a 2′,2′-difluoronucleoside of formula I, 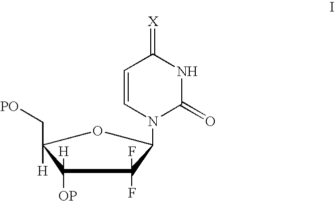
having an α/β ratio of about 1:4 to about 1:6 by HPLC, comprising combining a fluorinated protected sugar derivatives of formula II, 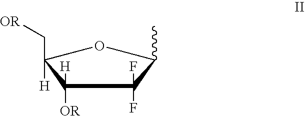
having an α/β ratio of about 1:1 to 1:2 as determined by HPLC, a water immiscible organic solvent and an organic base of formula III 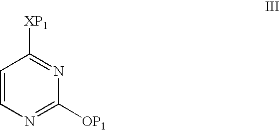
with a Lewis acid, to obtain a mixture. The mixture is then heated to a temperature of about 40° C. to about 140° C. until the conversion is of at least 80%, followed by quenching to give 2′,2′-difluoronucleoside of the formula I; wherein, L is a leaving group selected from the group consisting of C₁₋₁₀ alkyl, C₁₋₁₀ haloalkyl, C₁₋₁₀ aryl-esters, C₁₋₁₀ alkyl and C₁₋₁₀ aryl-sulphonates, and halogens; R is an alcohol protecting groups selected from the group consisting of C₁₋₁₀ alkyl- and C₁₋₁₀ aryl-ester ester, ether, carbamate and acetal; P₁ is a C₁₋₆ trialkyl silyl ether, wherein each alkyl group can be the same or different, and X is either NH and O.

In another aspect, the present invention provides a process for preparing Gemcitabine comprising preparing 2′,2′-difluoronucleoside of formula I as described above, and further converting it to Gemcitabine.

In yet another aspect, L in the process described above, is acetate group, R is a benzyl group and P₁ is trimethylsilyl group, and the obtained product is 3,5-dibenzoate-2,2-difluoro-uridine of the formula Ia.

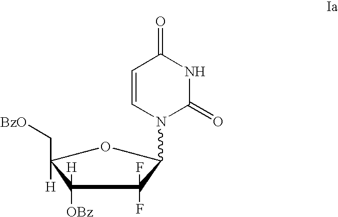

In one aspect, the present invention provides a process for preparing Gemcitabine comprising preparing 3,5-dibenzoate-2,2-difluoro-uridine of the formula Ia, as described above, and further converting it to Gemcitabine.

In another aspect, the present invention provides the novel compound, 2-deoxy-3,5-dibenzoate-2,2-difluoro-uridine of the formula Ia.

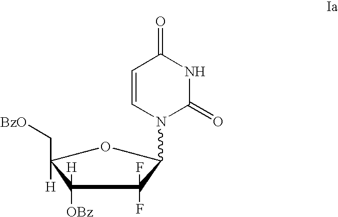

In yet another aspect, the present invention provides 2-deoxy-3,5-dibenzoate-2,2-difluoro-uridine of the formula Ia having α/β ratio of about 1:4 to about 1:6, as determined by HPLC.

In one aspect, the present invention provides the novel β isomer of 2-deoxy-3,5-dibenzoate-2,2-difluoro-uridine of the formula Ia-β, of the following structure.

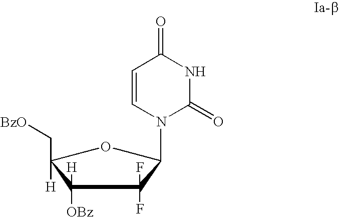

### BRIEF DESCRIPTION OF THE DRAWINGS

FIG. 1 illustrates the ¹H-NMR spectrum for a compound of formula Ia; and

FIG. 2 illustrates the ¹H-NMR spectrum for a compound of formula Ia-β.

### DETAILED DESCRIPTION OF THE INVENTION

The present invention provides a process to obtain Gemcitabine, by a stereoselective coupling reaction, which is done under mild condition, leading to the β enriched precursor of Gemcitabine, hence, avoiding purification steps such as, chromatography. Thus, the process of the present invention can be adapted to an industrial scale.

The present invention provides a process for the preparation of a 2′,2′-difluoronucleoside of formula I, 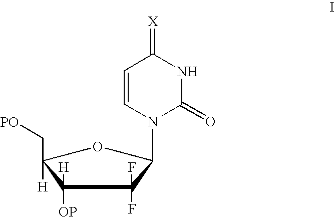
having an α/β ratio of about 1:4 to about 1:6 by HPLC, comprising combining a fluorinated protected sugar derivatives of formula II, 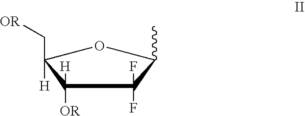
having α/β ratio of about 1:1 to 1:2, as determined by HPLC, a water immiscible organic solvent and an organic base of formula III 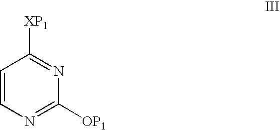
with a Lewis acid, to obtain a mixture. The mixture is then heated to a temperature of about 40° C. to about 140° C. until the conversion is of at least about 80%, followed by quenching to give 2′,2′-difluoronucleoside of the formula I; wherein, L is a leaving group selected from the group consisting of C₁₋₁₀ alkyl, C₁₋₁₀ haloalkyl, C₁₋₁₀ aryl-esters, C₁₋₁₀ alkyl and C₁₋₁₀ aryl-sulphonates, and halogens; R is an alcohol-protecting group selected from the group consisting of C₁₋₁₀ alkyl, C₁₋₁₀ aryl ester, ether, carbamate and acetal; P₁ is a C₁₋₆ trialkyl silyl ether, wherein each alkyl group can be the same or different, and X is either NH and O.

Preferably, the process of the invention may be used for the synthesis of 2′-deoxy-2′,2′-difluoroadenosine, 2′-deoxy-2′,2′-difluorouridine, 2′-deoxy-2′,2′-difluorothymidine, 2′-deoxy-2′,2′-difluoroguanosine, 2′-deoxy-2′,2′-difluorocytidine, and analogues thereof, which are obtained after a deprotection reaction of the protected 2′,2′-difluoronucleoside, obtained by the process of the present invention. The deprotection reaction may be done according to process known in the art, such as the ones described in J. Chem. Soc. Perkin Trans. I, 1982, 1171, J. Org. Chem., 1988, 53, 2406, Helv. Chim. Acta, 1995, 490 and in Org. Proc. Res. Dev., 2004, 8, 564

Preferably, R is either C₁₋₁₀ alkyl- or C₁₋₁₀ aryl-ester, more preferably, C₁₋₁₀ aryl-ester and most preferably, benzoyl ester. A more preferred P₁ is C₁₋₃ alkyl and most preferably, trimethylsilyl, Preferably, L is either C₁₋₁₀ alkyl, or C₁₋₁₀ aryl-esters, more preferably, C₁₋₁₀ alkyl ester, and most preferably, methylester.

The present invention further provides a process for preparing Gemcitabine comprising preparing 2′,2′-difluoronucleoside of formula I as described above, and further converting it to Gemcitabine.

The present invention also provides the process described above wherein, L is methyl ester and R is benzoyl ester, hence, the fluorinated protected sugar derivatives of formula II corresponds to 1-acetyl-2-deoxy-3,5-dibenzoate-2,2-difluoro-ribofuranose of the formula II-a, 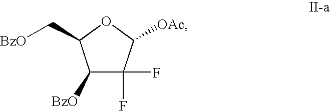
and wherein P₁ is trimethylsilyl group, hence, the organic base of formula III corresponds to 2,4-bis-O-trimethylsilyluracil of formula IIIa, 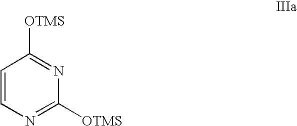
and the obtained 2′,2′-difluoronucleoside of formula I corresponds to 3,5-dibenzoate-2,2-difluoro-uridine of the formula Ia.

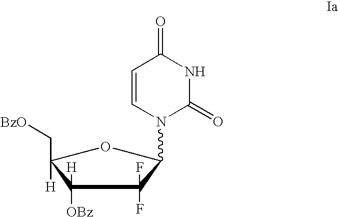

The 1-acetyl-2-deoxy-3,5-dibenzoate-2,2-difluoro-ribofuranose of formula IIa, may be prepared as exemplified in example 2. According to the process exemplified in example 3, the compound of formula IV, 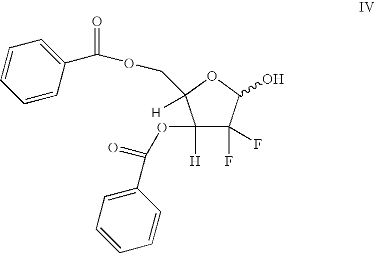
is combined with an organic base and an acetylating reagent, to obtain a mixture. The mixture is then maintained at a temperature of about 0° C. to about 40° C. for about 1 to about 18 h to give 1-acetyl-2-deoxy-3,5-dibenzoate-2,2-difluoro-ribofuranose, which is then recovered.

Preferably, the water immiscible organic solvent is selected from the group consisting of C₁₋₄ halogenated hydrocarbon, more preferably, either dichloroethane or dichloromethane, most preferably, dichloroethane.

Preferably, the organic base in the coupling step is commercial.

Preferably, the organic base in the coupling step is selected from the group consisting of pyrimidine and purine derivatives. Preferably, the pyrimidine derivative is cytosine, uracil or thymine. A preferred purine derivative is either guanine or adenine.

Preferably, the base is a protected base in which each oxygen atom is protected with a protecting group. Preferably, the base is a protected base in which each oxygen atom is protected with a protecting group. Preferably, the protected base is selected from the group consisting of 2-O-trimethylsilylcytosine, 2-O-trimethyl-N-trimethylsilylacetylcytosine, 2,4-bis-O-trimethylsilyluracil, 2,4-bis-O-trimethylsilylthymine, and 6-O-trimethylsilylguanine. Most preferably, the protected base is 2,4-bis-O-trimethylsilyluracil.

Preferably, the Lewis acid is TiCl₄, AlCl₃, BF₃, ZnCl₂, SnCl₂ or SnCl₄, more preferably, SnCl₄.

Preferably, the Lewis acid is used in an amount of 1.5 mole equivalent to 6 mole equivalent per mole equivalent of the compound of formula IV.

Preferably, the mixture is heated to a temperature of about 60° C. to about 120° C.

Preferably, the reaction is maintained at a temperature of about 60° C. to about 120° C. for about 1 to about 24 hours, preferably, for about 6 to about 24 hours until obtaining a conversion of at least 80%. Wherein, at this stage, the isomeric ratio is fixed, and the reaction can be stopped by quenching. Conveniently, the observed α/β ratio in 3,5-dibenzoate-2,2-difluoro-uridine of the formula Ia is not determined by the initial ratio of anomers in the starting sugar, but is driven by the nature of the catalyst and by the reaction solvent.

The conversion is preferably measured by HPLC.

Preferably, the mixture is cooled to a temperature of about 25° C. to about 20° C., prior to recovering of the product.

Preferably, quenching is done using a saturated aqueous solution of potassium or sodium bicarbonate, more preferably, potassium bicarbonate.

The 2-deoxy-3,5-dibenzoate-2,2-difluoro-uridine, of the formula Ia may be recovered from the reaction mixture by filtering the suspension obtained after quenching, followed by washing the filtrate with a saturated sodium bicarbonate solution and concentrating under reduced pressure.

The recovered 2-deoxy-3,5-dibenzoate-2,2-difluoro-uridine, of the formula Ia having an isomeric ratio of about 1:4 to about 1:6, determined by HPLC, is triturated in a mixture of heptane and ethyl acetate, in a ratio of 2 to 1 and filtered, to give 2-deoxy-3,5-dibenzoate-2,2-difluoro-uridine, of the formula Ia having an α/β ratio of about 2:98, as determined by HPLC.

The present invention provides a process for preparing Gemcitabine comprising preparing 3,5-dibenzoate-2,2-difluoro-uridine of the formula Ia, as described above, and further converting it to Gemcitabine, for example, according to processes known in the art, such as the ones described in J. Chem. Soc. Perkin Trans. I, 1982, 1171, J. Org. Chem., 1988, 53, 2406; Helv. Chim. Acta, 1995, 490 or in Org. Proc. Res. Dev., 2004, 8, 564.

The present invention further provides the novel compound, 2-deoxy-3,5-dibenzoate-2,2-difluoro-uridine of the formula Ia.

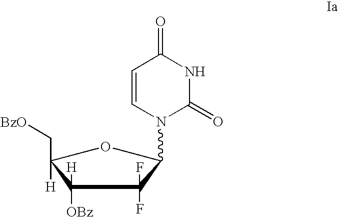

The present invention also provides 2-deoxy-3,5-dibenzoate-2,2-difluoro-uridine of the formula Ia having α/β ratio of about 1:4 to about 1:6, as determined by HPLC.

The 2-deoxy-3,5-dibenzoate-2,2-difluoro-uridine of the formula Ia of the present invention is characterized by an ¹H-NMR spectrum having peaks at about 4.85-4.55, 4.85, 5.25, 5.77, 5.95-5.80, 6.37, 6.60, 7.75-7.42, 7.90, 7.95-8.10 and 11.65 ppm. The

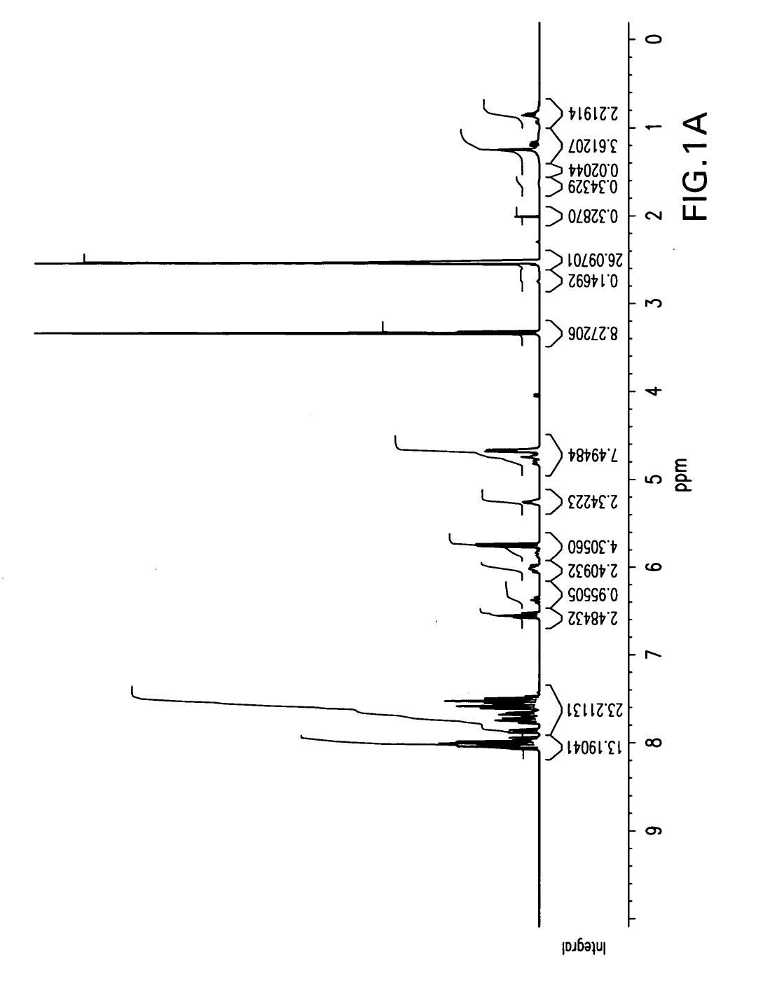

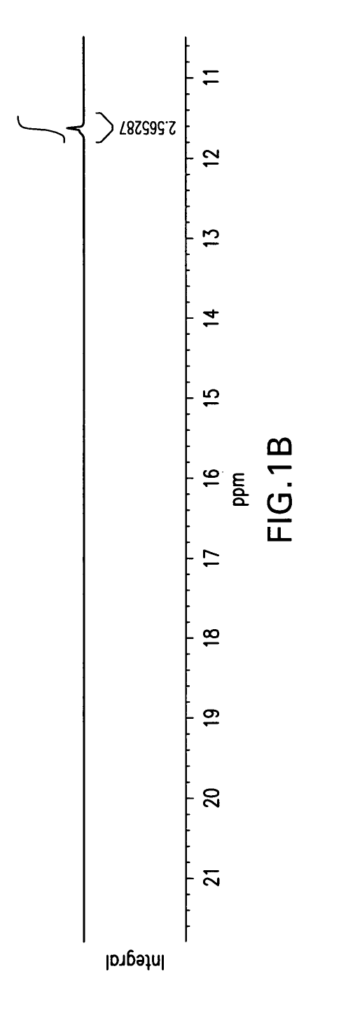

¹H-NMR spectrum for this compound is illustrated in FIG. 1.

The present invention provides the novel 0 isomer of 2-deoxy-3,5-dibenzoate-2,2-difluoro-uridine of the formula Ia-β, of the following structure.

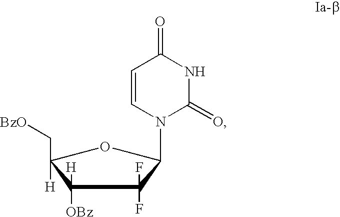

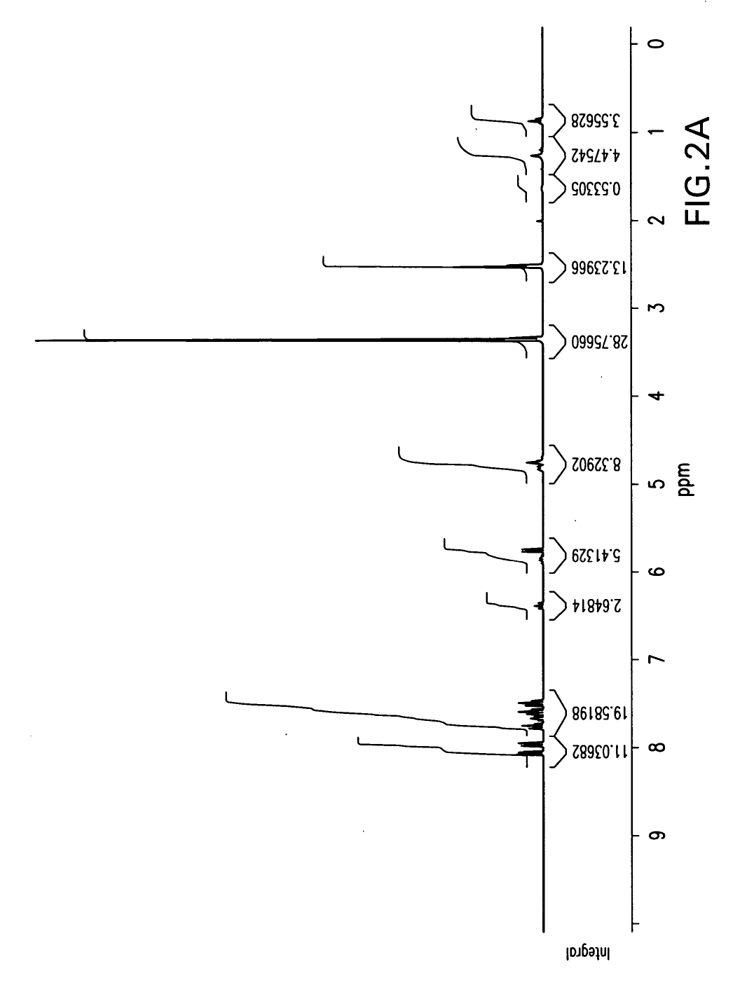

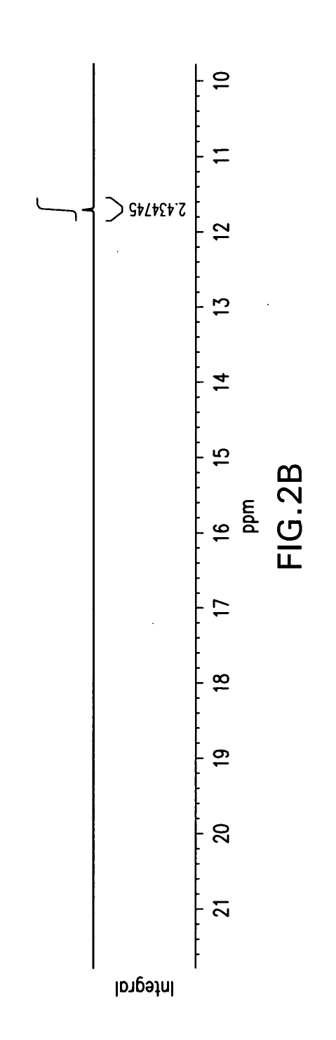

The β isomer of 2-deoxy-3,5-dibenzoate-2,2-difluoro-uridine of the formula Ia-β of the present invention is characterized by an ¹H-NMR spectrum having peaks at about 4.92-4.85, 5.77, 5,95-5.85, 6.37, 7.80-7.42, 7.90, 8.10 and 11.65 ppm. The ¹H-NMR spectrum of this compound is illustrated in FIG. 2.

### EXAMPLES

HPLC

The isomeric ratio was determined by the following HPLC method:

| Column & Packing: | HP Hypersil BDS-C 18 (125 * 4 mm) or | | | |
|---|---|---|---|---|
| | equivalent, | | | |
| Eluent A: | Acetonitrile (containing 0.1% TFA) | | | |
| Eluent B: | water | | | |
| | Time | | | Flow |
| Gradient conditions: | (minutes) | % Eluent A | % Eluent B | rate |
| | 0 | 1 | 99 | 1 ml/min |
| | 10 | 100 | 0 | 1 ml/min |
| | 12 | 100 | 0 | 1 ml/min |

| | |
|---|---|
| Detector: | 254 nm |
| Diluent: | acetonitrile |
| Sample | 2 mg/mL in acetonitrile |
| Concentration: | |

### Example 1

### A General Procedure for the Preparation of 2′,2′-difluoronucleoside of the Formula I

In accordance with the invention, the difluoro sugar derivative was dissolved in 20 to 30 volumes of solvent, then 1.5 to 4.5 equivalents of 2,4-bis-O-trimethylsilyluracil and 2 to 4.5 equivalents of Sn (II) or (IV) salts were added at room temperature. The mixture was heated at temperatures between 20° C. and 105° C., and the reaction was monitored by HPLC. When the desired conversion was observed, the mixture was cooled to room temperature, and then a saturated sodium bicarbonate solution was added. The mixture was filtered, and the filtrate was concentrated to dryness. Optionally, the crude mixture of stereoisomers was triturated in heptane/ethyl acetate and filtered to yield pure beta anomer as a white solid.

### Example 2

### 2-deoxy-3,5-dibenzoate-2,2-difluoro-uridine (from acetate)

A 0.46 g sample of 1-acetyl-2-deoxy-3,5-dibenzoate-2,2-difluoro-ribofuranose (compound IV that can be obtained from commercially available material e.g. by method reported in patent application WO2005095430) was dissolved in 15 ml of dichloroethane, and 1.26 g of 2,4-bis-O-trimethylsilyluracil and 0.89 ml of SnCl₄ were added at room temperature. The mixture was then heated to 83° C. for 22 hours. The mixture was then allowed to cool to room temperature, and quenched via addition of 20 ml of a saturated sodium bicarbonate solution. The suspension was filtered over a pad of Celite eluting with 100 ml of dichloromethane. The filtrate was washed with 20 ml of saturated sodium bicarbonate solution, dried over Na₂SO₄, and filtered and concentrated under reduced pressure to obtain an off-white foam.

The crude product, a 1:5 mixture of anomers, was triturated in a 2:1 heptane/ethyl acetate mixture, and filtered. The undissolved solid was identified (1H, 19F NMR, HPLC) as β-anomer (95% de) of the title compound.

¹H NMR: δ (300 MHz, DMSO): 11.65 (1H, s); 8.10 (2H, d); 7.90 (2H, d); 7.80-7.42 (7H, m); 6.37 (1H, t); 5.95-5.85 (1H, m); 5.77 (1, d); 4.92-4.85 (3H, m)

### Example 3

### 1-Acetyl-2-deoxy-3,5-dibenzoate-2,2-difluoro-ribofuranose

A 4.0 g sample of 2-deoxy-3,5-dibenzoate-2,2-difluoro-ribofuranose was dissolved in 40 ml of triethylamine, and 20 ml of acetic anhydride was added slowly. The mixture was stirred at room temperature for 17 hours, and then partitioned between 100 ml of dichloromethane and 40 ml of a saturated solution of sodium bicarbonate. The organic phase was dried over Na₂SO₄, and concentrated under reduced pressure. Chromatography of the residue over silica gel with a heptane/ethyl acetate eluent yielded the title compound (3.78 g, 1:1.19 mixture of anomers via ¹H, ¹⁹F NMR and HPLC), as a white solid.

δ (300 MHz, DMSO): 8.10-7.90 (4H, d); 7.85-7.50 (6H, d); 6.40 and 6.31 (1H, d); 6.00-5.90 (1H, m); 4.95-4.45 (3H, m); 2.18 and 2.00 (3H, s)

### Comparative Example 4

### Preparation of 2-Deoxy-3,5-dibenzoate-2,2-difluoro-1-methylsulphonyloxy-ribofuranose according to U.S. Pat. No. 5,594,124

A 4.0 g sample of 2-deoxy-3,5-dibenzoate-2,2-difluoro-ribofuranose was dissolved in 1.92 ml of triethylamine and 50 ml of dichloromethane. Then, 1.00 ml of methanesulphonyl chloride was added slowly. The resulting mixture was stirred at room temperature for 17 hours. The mixture was then partitioned between 100 ml of dichloromethane and 40 ml of a saturated solution of sodium bicarbonate. The organic phase was dried over Na₂SO₄, and concentrated under reduced pressure. Chromatography of the residue over silica gel (eluent heptane/ethyl acetate) yielded 4.91 g of the title compound in a 1:1 mixture of anomers via 1H, ¹⁹F NMR and HPLC, as a white solid.

### Comparative Example 5

### Preparation of 2-deoxy-3,5-dibenzoate-2,2-difluoro-uridine according to U.S. Pat. No. 4,965,374 (from mesylate):

2-Deoxy-3,5-dibenzoate-2,2-difluoro-1-methylsulphonyloxy-ribofuranose (500 mg) obtained as described above was dissolved in a pressure-proof vessel with dichloroethane (10 ml).

2,4-O-Bis-trimethylsilyluracil (420 mg) and trimethylsilyltriflate (0.297 mL) were added to the solution. The mixture was heated to 83° C. for 17 h, then cooled to 25° C. and partitioned twice between dichloromethane (40 mL) and saturated sodium bicarbonate solution (20 mL).

The combined organic extracts were dried over Na₂SO₄ and concentrated over reduced pressure to yield the crude product as an off-white foam (540 mg); α/β anomeric ratio (1.14/1, HPLC).

What is claimed is:

1. A process for the preparation of a 2′,2′-difluoronucleoside of formula I, 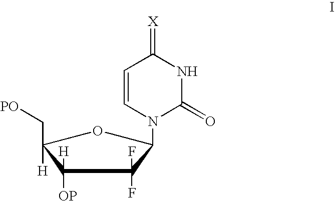
having an α/β ratio of about 1:4 to about 1:6 by HPLC, comprising
(a) combining a fluorinated protected sugar derivatives of formula II, 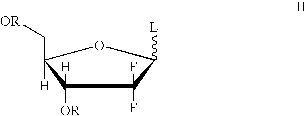
having an α/β ratio of about 1:1 to 1:2, as determined by HPLC, a water immiscible organic solvent and an organic base of formula III 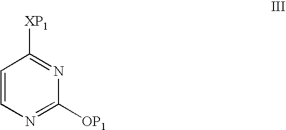
with a Lewis acid, to obtain a mixture;
(b) heating the mixture to a temperature of about 40° C. to about 140° C. until the conversion is of at least about 80%;
(c) quenching to give 2′,2′-difluoronucleoside of the formula I;
wherein, L is a leaving group selected from the group consisting of C₁₋₁₀ alkyl, C₁₋₁₀ haloalkyl, C₁₋₁₀ aryl-esters, C₁₋₁₀ alkyl and C₁₋₁₀ aryl-sulphonates, and halogens; R is an alcohol-protecting group selected from the group consisting of C₁₋₁₀ alkyl, C₁₋₁₀ aryl ester, ether, carbamate and acetal; P₁ is a C₁₋₆ trialkyl silyl ether, wherein each alkyl group can be the same or different, and X is either NH and O.

2. The process of claim 1, wherein the obtained 2′,2′-difluoronucleoside is 2′-deoxy-2′,2′-difluoroadenosine, 2′-deoxy-2′,2′-difluorouridine, 2′-deoxy-2′,2′-difluorothymidine, 2′-deoxy-2′,2′-difluoroguanosine, 2′-deoxy-2′,2′-difluorocytidine or analogues thereof.

3. The process of claim 1, wherein R is either C₁₋₁₀ alkyl- or C₁₋₁₀ aryl-ester.

4. The process of claim 3, wherein R is C₁₋₁₀ aryl-ester.

5. The process of claim 4, wherein R is benzyol ester.

6. The process of claim 1, wherein P₁ is C₁₋₆ trialkyl silyl ether.

7. The process of claim 6, wherein each alkyl group can be the same or different.

8. The process of claim 1, wherein P₁ is C₁₋₃ trialkyl silyl ether.

9. The process of claim 8, wherein the C₁₋₃ alkyl is methyl.

10. The process of claim 1, wherein L is either C₁₋₁₀ alkyl, or C₁₋₁₀ aryl-esters.

11. The process of claim 10, wherein L is C₁₋₁₀ alkyl ester.

12. The process of claim 11, wherein L is methyl ester.

13. A process for preparing Gemcitabine comprising
(a) combining a fluorinated protected sugar derivatives of formula II, having an α/β ratio of about 1:1 to 1:2, as determined by HPLC, a water immiscible organic solvent and an organic base of formula III with a Lewis acid, to obtain a mixture;
(b) heating the mixture to a temperature of about 40° C. to about 140° C. until the conversion is of at least about 80%;
(c) quenching to give 2′,2′-difluoronucleoside of the formula I;
(d) converting 2′,2′-difluoronucleoside of the formula I to Gemcitabine.

14. The process of claim 1, wherein L is methylester, R is benzoyl ester, P₁ is trimethylsilyl group and the obtained 2′,2′-difluoronucleoside of formula I corresponds to 3,5-dibenzoate-2,2-difluoro-uridine of the formula Ia.

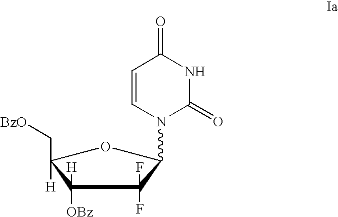

15. The process of claim 1, wherein the water immiscible organic solvent is selected from the group consisting of C₁₋₄ halogenated hydrocarbon.

16. The process of claim 15, wherein the C₁₋₄ halogenated hydrocarbon is either dichloroethane or dichloromethane.

17. The process of claim 15, wherein the C₁₋₄ halogenated hydrocarbon is dichloroethane.

18. The process of claim 1, wherein the organic base is selected from the group consisting of pyrimidine and purine derivatives.

19. The process of claim 15, wherein the pyrimidine derivative is cytosine, uracil or thymine.

20. The process of claim 15, wherein the purine derivative is either guanine or adenine.

21. The process of claim 15, wherein the base is a protected base in which each oxygen atom is protected with a protecting group.

22. The process of claim 21, wherein in the base each oxygen atom is protected with a protecting group.

23. The process of claim 22, wherein the protected base is selected from the group consisting of 2-O-trimethylsilylcytosine, 2-O-trimethyl-N-trimethylsilylacetylcytosine, 2,4-bis-O-trimethylsilyluracil, 2,4-bis-O-trimethylsilylthymine, and 6-O-trimethylsilylguanine.

24. The process of claim 23, wherein the protected base is 2,4-bis-O-trimethylsilyluracil.

25. The process of claim 1, wherein the Lewis acid is TiCl₄, AlCl₃, BF₃, ZnCl₂, SnCl₂ or SnCl₄.

26. The process of claim 25, wherein the Lewis acid is SnCl₄.

27. The process of claim 1, wherein the Lewis acid is used in an amount of 1.5 mole equivalent to 6 mole equivalent per mole equivalent of the compound of formula II.

28. The process of claim 1, wherein the mixture is heated to a temperature of about 60° C. to about 120° C.

29. The process of claim 1, wherein the reaction is maintained at a temperature of about 60° C. to about 120° C. for about 1 to about 24 hours.

30. The process of claim 1, wherein the reaction is maintained at a temperature of about 60° C. to about 120° C. for about 6 to about 24 hours.

31. The process of claim 1, wherein the mixture of step (d) is cooled to a temperature of about 25° C. to about 20° C., prior to quenching.

32. The process of claim 1, wherein quenching is done using a saturated aqueous solution of potassium or sodium bicarbonate.

33. The process of claim 1, wherein quenching is done using potassium bicarbonate.

34. The process of claim 1, further comprising triturating the recovered 2′,2′-difluoronucleoside of the formula I.

35. The process of claim 34, wherein the obtained 2′,2′-difluoronucleoside of the formula I has an α/β ratio of about 2:98, as determined by HPLC.

36. A process for preparing Gemcitabine comprising
(a) combining 1-acetyl-2-deoxy-3,5-dibenzoate-2,2-difluoro-ribofuranose of the formula II-a, 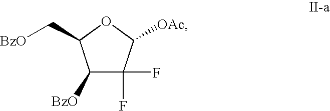
having α/β ratio of about 1:1 to 1:2, as determined by HPLC, a water immiscible organic solvent and an organic base, 2,4-bis-O-trimethylsilyluracil of formula IIIa, 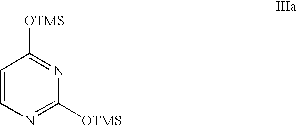
with a Lewis acid, to obtain a mixture;
(b) heating the mixture to a temperature of about 40° C. to about 140° C. until the conversion is of at least about 80%;
(c) quenching to give 3,5-dibenzoate-2,2-difluoro-uridine of the formula Ia;
(d) converting 3,5-dibenzoate-2,2-difluoro-uridine of the formula Ia to Gemcitabine.

37. 2-deoxy-3,5-dibenzoate-2,2-difluoro-uridine of the formula Ia.

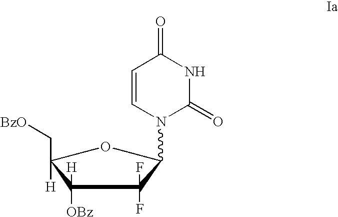

38. 2-deoxy-3,5-dibenzoate-2,2-difluoro-uridine of the formula Ia having an α/β ratio of about 1:4 to about 1:6, as determined by HPLC.

39. The 2-deoxy-3,5-dibenzoate-2,2-difluoro-uridine of the formula Ia of claim 38

40. β isomer of 2-deoxy-3,5-dibenzoate-2,2-difluoro-uridine of the formula Ia-β.

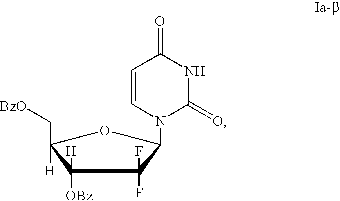

41. The β isomer of 2-deoxy-3,5-dibenzoate-2,2-difluoro-uridine of the formula Ia-β of claim 40.
